(19) United States
(12) Patent Application Publication
(10) Pub. No.: US 2008/0281000 A1
(43) Pub. Date: Nov. 13, 2008

(54) Hydrophobic Polysaccharide Derivatives

(76) Inventors: William H. Daly, Baton Rouge, LA (US); Ahmad A. Bahamdan, Dhahran (SA)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/065,846

(22) PCT Filed: Sep. 6, 2006
(86) PCT No.: PCT/US06/34577

§ 371 Date: Mar. 5, 2008

Related U.S. Application Data
(60) Provisional application No. 60/715,757, filed on Sep. 8, 2005.

(51) Int. Cl. C08B 37/00; C08B 37/08; C08B 11/00; A61K 47/26; A61P 27/02; C09K 8/68; C08B 31/08

(52) U.S. Cl. 514/778; 536/20; 536/43; 536/50; 536/52; 507/211; 507/212; 507/213; 507/214; 514/777; 514/781

(57) ABSTRACT

Novel cross-linked gels comprised of alkoxyetheramides grafted to polysaccharides that have superior viscosity properties have been made. By controlling the chain length of the alkoxyetheramides and the hydrophobic nature of the gel, these materials are ideal for many uses such as in hydraulic fracturing of oil-bearing geological formations, in the paint and dye industries, as dispersants, in personal care products and for carriers in controlled drug delivery.

(In countries other than the United States:) The benefit of the 8 Sep. 2005 filing date of U.S. patent application Ser. No. 60/715,757 is claimed under applicable treaties and conventions. (In the United States:) The benefit of the 8 Sep. 2005 filing date of provisional patent application No. 60/715,757 is claimed under 35 U.S.C. § 119(e).

### TECHNICAL FIELD

This invention pertains to a process for forming a viscous gel and then, at a time selected by a user, fragmenting the gel into fragments with surfactant properties. This invention also pertains to polymers useful in the gel compositions. The invention may be used, for example, in hydraulic fracturing of oil-bearing geological formations, in paints and dyes, as dispersants, in personal care products, and for carriers in controlled drug delivery.

### BACKGROUND ART

Polysaccharides, such as guar powder, carboxymethyl guar, cellulose, starch, chitin, chitosan, polygalactomannan, polyglucomannan, galactomannan gum, and xanthan, and polyvinyl alcohol have been derivatized in efforts to form grafts that have low enough initial viscosity to be handled easily and that will cross-link in situ to form higher viscosity fluids that also can be broken in situ.

Much of this work has been done on guar gum. Guar gum is a naturally occurring, non-ionic, hydrophilic polygalactomannan polysaccharide derived from the seed of the guar plant. The chemical structure of guar gum, as seen in FIG. 1, comprises anhydro-D-mannose monomer units linked to one another by β-(1→4) linkages to form the backbone chain. Anhydro-D-galactose branches are joined by α-(1→6) bonds to the backbone; on average, the galactose branches occur on alternate mannose units. Guar gum has a molecular weight of about 2×10⁶ Da, and can be dispersed into water and brines. Guar gum exhibits non-Newtonian viscosity, and suspensions of guar gum in water can be cross-linked to give very high strength gels. (Jasinski, Raymond; Redwine, David and Rose, Gene, Journal of Polymer Science: Part B: Polymer Physics, 1996, 34, 1477-1488.) While guar gums hydrate well in aqueous solutions, they often exhibit neither solution clarity, solubility in alcohols, nor good thermal stability. Thus a number of chemically modified guar gums have been developed. (Moorhouse, Ralph, Harry, David N., and Merchant, Uday, Society of Petroleum Engineering, SPE 39531, 1998, 253-269.)

An average of three hydroxyl groups per anhydroglucose unit is available for derivatization in guar gum. The maximum theoretical degree of substitution (“DS”) in such molecules is thus three. When alkene oxides, for example propylene oxide, react with available hydroxyl groups to form hydroxylalkyl substituents, new hydroxyl groups become available for further additions, so that a side chain may be extended. Molar substitution (“MS”) is then defined as the average number of alkene oxide units attached per sugar unit. Therefore MS can exceed three.

Widely used derivatives of guar gum include CarboxyMethyl-Guar (CMG), HydroxyPropyl-Guar (HPG), and CarboxyMethylHydroxyPropyl-Guar (CMHPG), as shown in Table 1. These derivatives also have been used as the polysaccharide component in certain grafts and gels.

TABLE 1
Guar Gum Derivatives

| | Structure of | |
|---|---|---|
| Type of Derivative | Substituent | Ionic Charge |
| Hydroxypropylguar (HPG) | —CH₂—CH(OH)CH₃ | Nonionic |
| Carboxymethylguar (CMG), | —CH₂—COO⁻Na⁺ | Anionic |
| Carboxymethylhydroxypropylguar | —CH₂—COO⁻Na⁺ | Anionic |
| (CMHPG) | —CH₂—CH(OH)CH₃ | |

Cross Linking Agents

High viscosity may be attained in grafts either by increasing the polymer concentration or by cross-linking the polymer molecules. Increasing the polymer concentration is normally not cost-effective and may cause operational problems. While low concentrations of guar gum (e.g., 0.3-0.5%) dissolved or suspended in water significantly increase the viscosity of the fluid (e.g., from 1 to 150 cP), the addition of millimolar amounts of cross-linking agents, such as borate ions, into a guar gum solution increases the viscosity several orders of magnitude (e.g., from 150 cP to 5700 cP). Cross-linking between guar and polyvalent hydroxyl complexes i.e., Ti, Zr, Al, Cr has also been observed. While not wishing to be bound by this theory, it is generally believed that the increase in viscosity for boron cross-linking is due to borate linking to two guar units at adjacent cis-hydroxy groups on the mannose backbone.

Borate and zirconate crosslinking systems are widely used. Zirconate can be used at a wider range of pHs, while borate systems are used at pHs from 9-11 only. Borates were once used for low temperature applications only (around 100° C.), while zirconate systems were generally used for higher temperature applications. However, borate-crosslinked HPG fracturing fluid systems at high temperatures (>149° C.) have also been reported. Again not wishing to be bound by this theory, it appears that zirconates, like borates, form chemical bonds with cis-OH groups on the polymer chains. Unlike borates, zirconate-crosslinked bonds are irreversible. Thus, the degradation of a zirconate-crosslinked gel requires high shear or cleavage of the polysaccharide backbone. Stabilizers are used at elevated temperatures to control the degradation of polysaccharides such as guar, HPG and CMHPG. Among the most common gel stabilizers are sodium thiosulfate and methanol. Again not wishing to be bound by this theory, it appears that these stabilizers act as reducing agents to inhibit the “unzipping” of oxidized polymer chains.

Hydrophobically Modified Guar Gum

Hydrophobically modified guar gum (“HMGG”) has been used as an alternate to the crosslinked guar gum technology. Incorporation of short chain hydrocarbon substituents using hexadecyl epoxides has led to polymers possessing both hydrophilic and hydrophobic parts. Though not wishing to be bound by this theory, in appears that the mechanism for aggregation of these nonionic polysaccharides is similar to that for surfactant micelles, i.e. minimizing contact between alkyl chains and surrounding water molecules, which drives the compound toward self-association. HMGGs are expected to be very efficient viscosifiers in aqueous media. Their viscosities increase with increasing hydrophobic content and with alkyl chain length, provided that the hydrophobic/hydrophilic balance is controlled to assure water solubility. The dissociated HMGGs exhibit lower hydrodynamic volumes because the dissociated products contain single chains rather than polymer complexes. Further reduction in the molecular weight of HMGGs can be achieved by acid hydrolysis, which produces shorter chain block copolymers, which may act as polymeric surfactants.

There are two general ways to graft onto a polymer backbone. “Grafting from” a backbone refers to methods of growing a graft from an activated site on the backbone. “Grafting to” refers to processes based upon attaching preformed polymers to active sites on the backbone.

Young et al. reported that hydrophobically modified hydroxybutyl guar (MHBG) shows improved rheological properties over native guar, hydroxypropyl guar (HPG) and hydroxybutyl guar (HBG). (Young, N. W. G.; Williams, P. A.; Meadows, J, and Allen, E., Society of Petroleum Engineering, SPE 39700, 1998, 463-470.)

Graft copolymers of hydrophobic monomers produce polymeric micelles and aggregates in an aqueous environment, which may be dispersed in aqueous/non-aqueous mixtures. A graft copolymer of methylacrylamide (MAM) grown from a guar gum backbone, using a potassium chromate/malonic acid redox pair, was reported by K. Behari and coworkers (Behari, K.; Kumar, R.; Tripathi, M. and Pandey, P. K., Macromol. Chem. Phys., 2001, 202, 1873-1877).

P. Chowdhury, et al. reported grafting methyl methacrylate (MMA) from guar gum utilizing a ceric ammonium sulfate/dextrose redox pair (CAS/DM). (Chowdhury, P.; Samui, S.; Kundu, T. and Nandi, M. M., Journal of Applied Polymer Science, 2001, 82, 3520-3525.)

Grafting acrylic acid (AA) from guar gum was reported by K. Taunk, et al. using a potassium peroxydiphosphate (PDP)/silver nitrate redox system. (Taunk, Kavita and Behari, Kunj, Journal of Applied Polymer Science, 2000, 77, 39-44.)

Grafting polyacrylonitrile from guar gum with a potassium persulfate/ascorbic acid redox system was reported by Bajpai, U. D. N. Mishra, Veena., and Rai, Sandeep, Journal of Applied Polymer Science, 1993, 47, 717-722.

To our knowledge, there have been no prior reports of graft copolymers in which the graft chain length was systematically controlled, allowing control over the properties of the graft. There is unfilled need to develop grafts whose properties may be systematically controlled.

Gel Breakers

Gel breakers are used to undo cross-linking where desired. It is important that gel breakers allow the system to maintain high viscosity in initial use, while allowing the breaking of the cross-linking and cleaving of backbone chains at an appropriate time to return to a lower fluid viscosity. Most of the systems used to date have exhibited a rapid initial drop in viscosity followed by a rather gradual decline in viscosity until the fluid is completely broken. There is an unfilled need for better control over the breaking process.

Breakers used with fracturing fluids fall into two general classifications: oxidizers and enzymes. Oxidizers, such as persulfates, are effective from about 50°-80° C., but at higher temperatures these materials react too quickly and cause uncontrolled breaking and premature gel degradation. Encapsulation of the breaker may provide a slower release, improving the break profile at low and moderate temperatures (around 95° C.), but at higher temperatures this method provides little improvement.

Enzymes are typically limited to lower temperatures (<65° C.) and limited to a pH range of 5 to 8. The use of encapsulation may provide a slight improvement in the stability of enzymes.

Guar gum grafts may be used, for example, as carrier fluids for enhanced oil recovery operations (“EOR”). An established technique for EOR is Hydraulic Fracturing Technology (“HFT”). While HFT has been used in the oil and gas industry for many years, the key to its effectiveness is the carrier fluid. Carrier fluids ideally will have a high initial viscosity that can be reduced (to facilitate efficient removal of the carrier) after a “proppant” is in place. This technique involves using high pressure to pump a slurry containing a proppant into a restrictive formation, creating new cracks and expanding existing cracks. The proppant remains in the structure to keep the cracks open when the external pressure is released, as the carrier liquid is removed. Highly viscous liquids are used as carriers to keep a proppant suspended during injection. However, if the carrier is too viscous then the carrier becomes difficult to handle. Further, after the proppant is in place, the carrier liquid must be “broken” in order that it can be readily and quantitatively removed, so the oil flow is not inhibited. To the knowledge of the inventors, no one has previously disclosed a system with sufficiently high initial viscosity that also can be readily and quantitatively removed from a formation at a selected time.

U.S. Pat. No. 6,810,959 discloses a low residue well treatment fluid comprising an aqueous solvent; a gelling agent comprising one or more modified polysaccharides, the modified polysaccharides having hydrophilic groups; and a crosslinking composition. The fluid may optionally further comprise a gel breaker, a buffer or a “proppant.” The fluids generate no, or minimal, residue upon being broken, and were described as being useful in well fracturing operations. The polysaccharides are modified with cationic or amphoteric hydrophobic groups.

U.S. Pat. No. 6,387,853 discloses derivatized polymers that may be introduced into a well bore, such as in hydraulic fracturing. The polymer may be a guar powder, carboxymethyl guar, cellulose, starch, polyvinyl alcohol, polygalactomannans, polyglucomannans, galactomannan gums, xanthans, and derivatives. The polymer is mixed with an organic solvent and derivatized using an agent such as sodium chloroacetate. The polymer is typically derivatized in bulk prior to introduction into the well bore. The derivatized polymer may be hydrated or cross-linked prior to introduction into the well bore. Derivatizing agents included alkylene oxide, alkali metal haloacetate, and haloacetic acid; such as sodium chloroacetate, sodium bromoacetate, chloroacetic acid, bromoacetic acid, or propylene oxide. The resulting derivatized polymers are either carboxymethyl or hydropropyl modified polymers and are not considered to be graft copolymers.

Q. Gu et al., “Enzyme-catalyzed esterification of cellulosics, guar, and polyethers,” Polymer Preprints, vol. 46, pp. 30-31 (2005) discloses the use of enzymes such as lipases to prepare substituted celluloses or guars, or to prepare alkyl ketene dimer derivatives of cellulose derivatives, guar, or poly(ethylene glycol).

U.S. Pat. No. 6,737,386 discloses a high pH aqueous zirconium crosslinked guar fractioning fluid suitable for use in petroleum wells operating at high temperature.

### SUMMARY OF THE INVENTION

We have discovered a novel process for forming a viscous gel, and then at a time selected by a user, fragmenting the gel into fragments with surfactant properties. We have also discovered novel gels comprising alkoxyethers grafted to polysaccharides. Previously, systematic control of the length of a polymer grafted to a polysaccharide has not been possible. By grafting polymers to a polysaccharide we have controlled the size and properties of gels, and enabled the gels to crosslink. The materials had sufficiently low viscosity to be handled easily before crosslinking. After crosslinking, they exhibited sufficiently high viscosity to be useful as a carrier in applications such as fracturing fluid for enhanced oil recovery, for medicines, for personal products, and other applications. The novel cross-linked gels may be degraded when desired to low molecular weight fragments having surfactant properties.

The polysaccharide component may, for example, be selected from guar, cellulose, starch, chitin, chitosan, polygalactomannan, polyglucomannan, galactomannan gum, xanthan, and derivatives of these compounds. The alkoxyether component may be chosen from alkylaryloxypoly(oxyalkylene)amides or alkoxypoly(oxyalkylene)amides. Preferred gels are polyoxyalkyleneamides grafted to guar gum and its derivatives, such as carboxymethylguar (“CMG”) or carboxymethylhydroxypropylguar (“CMHPG”). We have discovered that for polyoxyalkyleneamides containing both polyoxyethylene and polyoxypropylene moieties, the ratio of polyoxyethylene ether groups to polyoxypropylene ether groups determined the hydrophobicity, solubility and the overall characteristics of the final gels.

### BRIEF DESCRIPTION OF THE DRAWINGS

FIG. 1 depicts the basic chemical structure of guar gum.

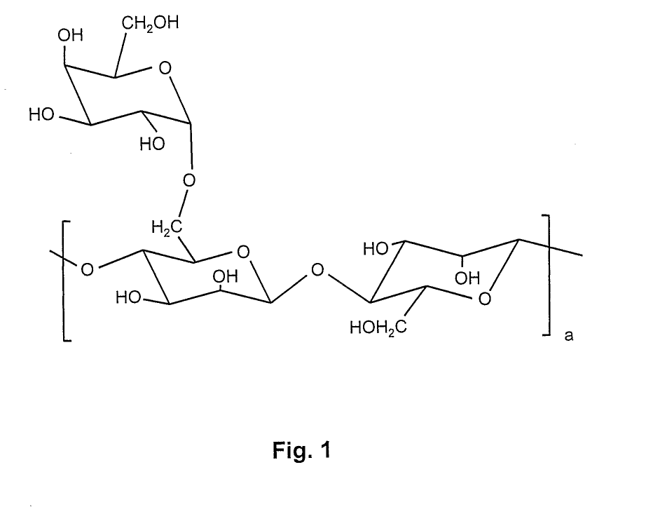

FIG. 2 depicts the synthesis of guar gum polyoxyalkyleneamine derivatives.

### MODES FOR CARRYING OUT THE INVENTION

Polysaccharides used in prototype experiments are listed in Table 1. These compounds were uncharged, water-soluble polysaccharides with the ability to crosslink readily with crosslinking agents. Crosslinking agents may, for example, be selected from boric acid, borate salts, zirconates, ZrOCl₂, zirconium lactate, zirconium glycolate, zirconium lactate triethanolamine, zirconium acetylacetonate, Zr chelates, titania, titanates, titanium citrate, titanium malate, titanium tartrate, Ti chelates, and aluminates. Zirconium lactate is a preferred crosslinking agent. Gels were formed by adding the crosslinking agent to low-viscosity polysaccharide solutions in situ.

The alkoxyalkyleneethers used in prototype demonstrations are listed in Table 2.

TABLE 2
Names and structures of polyoxyalkyleneamines disclosed herein.

| | | Ratio | |
|---|---|---|---|
| | | PO/EO | Approximate |
| Polyoxyalkyleneamine | Structure | (y/x) | Mol. Wt. |
| XTJ-505 (M-600) | CH₃—[OCH₂—CH2]ₓ—[OCH₂—CH(CH3)]_{y}—NH₂ | 9/1 | 600 |
| Jeffamine M-715 | CH₃—[OCH₂—CH2]ₓ—[OCH₂—CH(CH3)]_{y}—NH₂ | 2/11 | 715 |
| XTJ-506 (M-1000) | CH₃—[OCH₂—CH2]ₓ—[OCH₂—CH(CH3)]_{y}—NH₂ | 3/19 | 1000 |
| Surfonamine L-300 | CH₃—[OCH₂—CH2]ₓ—[OCH₂—CH(CH3)]_{y}—NH₂ | 8/58 | 3000 |
| Surfonamine MNPA- | C₉H₁₉—C₆H₄—O—[CH₂CH(CH₃)]_{12.5}—OCH₂CH(CH₃)—NH₂ | — | 1004 |
| 1000 (B100) | | | |
| Surfonamine ML-300 | CH₃(CH₂)₁₂OCH₂CH(CH3)—OCH₂CH(CH₃)—NH₂ | — | 325 |
| (B30) | | | |

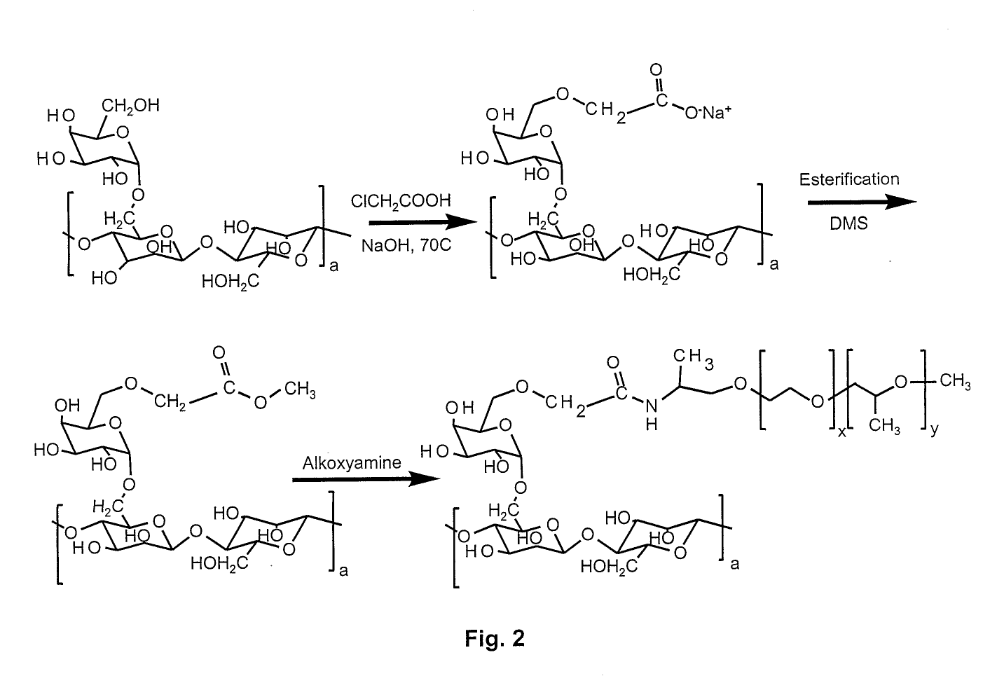

Table 2 also shows the ratios of the number of repeating oxypropenyl units, y, to the number of repeating oxyethylenyl units, x, and the molecular weight of the polymers. We used a family of amine derivatives, amine-terminated poly(ethyleneoxide-co-propyleneoxide) oligomers (PEO-PPO-NH₂), to modify guar gum. Other polymers may be used in place of guar gum. Since the molecular weights and molecular weight distributions of the oligomers were well defined, we were able to generate grafts onto carboxymethylated guar substrates of controlled lengths, compositions and properties. We have varied oligomer molecular weights, compositions, and degree of guar carboxymethylation to produce a series of guar derivatives with a range of features, viscosities and potential applications. A reaction scheme showing this approach is depicted in FIG. 2.

In a preferred embodiment, guar gum was modified with side chains to impart surfactant character to the fragments once the gel was broken. Hydrophobic groups such as alkoxypoly(oxyalkene) groups were conjugated to the guar gum with a preferred embodiment using alkoxy(polyoxyalkylene)amides for the grafting group Other hydrophobic groups such as amine terminated polyvinyl oligomers (e.g., polyvinyl oligomers of styrenes), acrylates (e.g., methacrylate, butylacrylate, laurylacrylate), and vinyl pyridines may be used, and other bean gum polysaccharide derivatives may be used. Following fragmentation, the substituted oligosaccharide fragments acted as surfactant molecules, with the sugar end acting as the hydrophilic portion of the surfactant molecule, and the substituents (e.g., alkoxopoly(oxyalkene)s acting as the hydrophobic portion.

We also have found the degree of substitution and the size of the side groups to be important factors. If the degree of substitution, or the size of the group, was too small, then the fragments had insufficient surfactant character. However, if the degree of substitution, or the size of the group, was too large, then the side groups sterically interfered with crosslinking, leading to an unsatisfactory gel. A useful range for the degree of substitution (number of substituents per saccharide unit) was between about 0.05 and about 0.5, preferably between about 0.15 and about 0.25. The side groups had molecular weights between about 250 and about 3000 Daltons, preferably between about 300 and about 1000 Daltons.

Cross-linking the substituted guar gum or other polysaccharides may employ methods otherwise known in the art for cross-linking, using agents such as using borates, zirconates, ZrOCl₂, zirconium lactate, zirconium glycolate, zirconium lactate triethanolamine, zirconium acetylacetonate, Zr chelates, titania, titanates, titanium citrate, titanium malate, titanium tartrate, Ti chelates, and aluminates or other crosslinkers. In a preferred embodiment, zirconium lactate was used to cause cross-linking.

“Breaking” the cross-linked gel may employ methods otherwise known in the art using agents such as enzymatic breaking, oxidative breaking using agents such as peroxides, persulfates, perborates, oxyacids, and oxyanions of halogens, or reductive breaking using agents such as Cu⁺²-chelated EDTA, aminocarboxylates, diamines, FeCl₂ and FeCl₃. In a preferred embodiment, mixtures of enzymes such Pyrolase™ 200 were used as breaking agents.

Listed below are prototype gels we have synthesized. The gel name is a combination of the names of the polysaccharide and the polyoxyalkyleneamide used. For example, a graft of XTJ-506 (M-1000) onto Carboxymethylguar is named “CMG-M1000.”

| | |
|---|---|
| CMG Control | CMHPG- |
| | MNPA1000 |
| CMG-M600 | CMHPG-L300 |
| CMG-M1000 | CMHPG-B30 |
| CMG-L300 | CMHPG-M600 |
| CMG-MNPA1000 | CMHPG-M715 |
| CMG-B30 | CMHPG-M1000 |
| CMHPG-Control | CMHPG- |
| | MNPA1000 |
| CMHPG-M600 | CMHPG-L300 |
| CMHPG-M715 | CMHPG-B30 |
| CMHPG-M1000 | |

Methods for Using the Gels

One application for these gels is as a carrier for hydraulic fracturing fluids for petroleum-bearing geological formations. In hydraulic fracturing, a liquid (typically aqueous) is pumped into a formation at high pressure, creating new cracks and causing existing cracks to expand. To inhibit the collapse of the cracks when the pressure is subsequently reduced, a “proppant” such as sand is pumped into the formation along with the liquid. The proppant particles move into cracks and help keep the cracks open when the external pressure is released. Oil then flows more readily through the formation.

The carrier liquid used in the fracturing must be highly viscous to inhibit settling of the sand or other proppant. However, once the proppant is in place, the carrier liquid needs to be removed so that oil flow is not inhibited. There is an unfilled need for a carrier fluid that is easy to handle, while sufficiently viscous to support the proppant during pumping. Further, the carrier's viscosity must be readily reduced after the proppants are in place, so that in the carrier liquid may be readily and quantitatively removed from the formation. This invention provides a carrier fluid with these properties. The gel precursors were of sufficiently low initial viscosity to be easily handled before crosslinking, but of sufficiently high gel viscosity to be effective carrier fluids. Further, these gels readily crosslinked, but were also readily and quantitatively broken, as needed. The fragments resulting from breaking were hydrophobic and soluble in organic liquids. Thus these gels had suitable characteristics for use in enhanced oil recovery.

The gels may also be used in medical treatment, e.g., ophthalmic treatment. For example, a gel may be formed in situ by injecting the carbohydrate and the cross-linking agent through concentric bores of a hypodermic needle, roughly analogous to the dispensers that are sometimes used for epoxy cements. In another application, a gel containing a drug may be placed onto the surface of the retina with this technique. This technique allows a therapeutic agent to be administered to the retina in a single injection, rather than in multiple injections as is now typically done when drugs must be administered to the retina. Furthermore, if desired, the gel could be hydrolyzed or broken after the medicine was in place.

### EXAMPLE 1

### Materials

Guar gum was provided by Dowell Schlumberger. Carboxymethylhydroxypropyl (CMHPG) and carboxymethyl guar gels (CMG) were provided by Benchmark. Polyoxyalkyleneamines, sold under the trade names Jeffamines and Surfonamines, were supplied by Texaco and Huntsman Chemical respectively. Chloroacetic acid (CAA) and CDCl₃ were purchased from Aldrich, and dimethylsulfate and sodium chloroacetate (SCA) were purchased from Acros. These reagents were used without further purification. All other chemicals were purchased from either Aldrich or Acros.

### EXAMPLE 2

### Instrumentation

Infrared spectra were obtained with a Bruker Tensor 27 series Fourier transform infrared (FT-IR) spectrometer using a horizontal attenuated total reflectance accessory (HATR) at 4 cm⁻¹ resolution and 16 scans.

### EXAMPLE 3

Nuclear Magnetic Resonance (NMR) analyses were performed using Bruker NMR DPX250 and DPX 300.

### EXAMPLE 4

Viscosity measurements on soluble graft copolymers were taken using a Brookfield RVT Dial Reading Viscometer Model, with a number 4 spindle. The poly(oxyalkylene)amide grafts to guar were left to hydrate for at least 1 hour before any measurements were performed. At least five revolutions were allowed to pass before recording any dial reading. The viscosity was measured at different rotation speeds from 0.5 to 100 rpm. Three readings were taken at each speed. Viscosities were calculated by multiplying the average dial reading by a conversion factor supplied by the manufacturer, and reported in cP.

### EXAMPLE 5

Viscosity measurements on cross-linked gels were performed using a Fann Model 35A viscometer (F-1 model) equipped with a heating cup capable of heating the fluids to 200° F. (93° C.). Gel viscosities were measured at room temperature and 65° C. with a B2 bob and R1 rotor, which allowed testing of cross-linked fluids. For higher temperatures (90° C. and 120° C.), a Brookfield PVS rheometer equipped with B5 bob was used. The sample chamber of the PVS instrument was capable of pressures up to 1000 psi and temperatures greater than 250° C. Both devices functioned as couette coaxial cylinder rotational viscometers.

The Fann Model 35A viscometer had a shear rate constant, K₃, of 0.377 sec−¹/RPM, which is used to calculate the shear rate by

Shear Rate=K₃*N
where N is the speed in rpm
   The viscosity is calculated as:

Viscosity (cP)=R*S*C*f

where:
R: is the dial reading
f: is the spring factor
S: is a speed factor (instruction manual)
C: the rotor-bob factor.

### EXAMPLE 6

### Viscosity Measurement

Polymer solutions (0.48 wt %) were prepared by dispersing the polymers in deionized water in concentrations from about 4.8 g/L (40 lb/1000 gal) to about 2.4 g/L (20 lb/1000 gal). The gels were left to hydrate at least 30 min. Sodium thiosulfate (1.2 g/L (10 lb/1000 gal)) was added as a gel stabilizer, and in some cases we used deionized water containing 4.61×10⁻⁴ g/L sodium azide to stop microorganism growth. The viscosity of the polymer fluid before cross-linking (linear gel) was measured at different shear rates with the Farm 35A (0.7 s⁻¹, 1.1 s⁻¹, 2.3 s⁻¹, 11.3 s⁻¹, 22.6 s⁻¹, 37.7 s⁻¹, 113 s⁻¹, and 226 s⁻¹) using the B2 bob and R1 rotor configuration.

### EXAMPLE 7

### Preparation of Sodium Carboxymethyl Guar (NaCMG) from Sodium Chloroacetate

NaCMG was synthesized under heterogeneous conditions following a slight modification of the method of Schult, T. and Moe, S. T., 9^{th} International Symposium on Wood and Pulping Chemistry, Vol. 2, pp. 99-1 through 99-4 for the synthesis of carboxymethyl cellulose. A slurry of guar gum, 70 g, was stirred in 400 mL of 2-propanol under nitrogen and allowed to swell for 30 min. A NaOH solution (40% w/w) (24.8 g) was added, and the mixture was held at room temperature to allow further swelling. 60 g of an aqueous solution of sodium chloroacetate (40% w/w) was then added, and the mixture was allowed to react for 1 hour at room temperature. The temperature of the reaction was then slowly raised to 70° C. and held there for 2-3 hours. The mixture was filtered after cooling to room temperature. The solid filtrate was washed twice with 400 mL of methanol/water (80% v/v), and then the filtrate was washed with methanol followed by acetone. The resulting solid product was dried at 60° C. overnight.

### EXAMPLE 8

### Determination of the Degree of Substitution Via Titration

The initial degree of substitution of CMG and CMHPG was measured by a modification of the titration method of ASTM D 1439. (ASTM D 1439, Vol. 6.03 (1994).) The sodium salt of CMG or CMHPG (10 g) was slurred with stirring into 150 mL of ethyl alcohol (95%); then 6-12 mL of 70% HNO₃ (sp.gr. 1.42) was added and stirred for 20 minutes. While stirring, the slurry was heated to boil, kept there for 5 min., and then the heat was removed. The stirring continued for 20 additional minutes. The mixture was filtered; the filtrate was added to 150 mL methanol (80%), stirred for 15-20 min, and then filtered, to remove salts and excess acid. This washing process was repeated three times. The resultant acid form of CMG or CMHPG was washed with methanol and dried at 60° C. overnight.

A 1 g aliquot of the dried acid form of CMG or CMHPG was transferred to a 200 mL Erlenmeyer flask and dissolved in distilled water (100 mL). An excess of 0.5 N NaOH (10-15 mL) solution was added with stirring and left to stir for 15 minutes. The solution temperature was raised to boiling and held there for 15-30 min. The excess NaOH was titrated with 0.5 N HCl to a phenolphthalein end point while the solution was hot.

The degree of substitution (“DS”) was determined according to the following expression:

DS=0.162A/(1−0.058A)

where:
A=(BC−DE)/F
A=acid consumed per gram of sample
B=NaOH (mL) solution added
C=normality of NaOH
D=HCl (mL) required to titration excess NaOH
E=normality of HCl
F=mass of CMG(g)
162=gram molecular mass of an anhydroglucose unit of CMG, and
58=net increase in molecular mass of an anhydroglucose unit for each carboxymethyl group substituted.

### EXAMPLE 9

Carboxymethylation of guar was adjusted to produce different degrees of substitution by altering the amounts and the molar ratio of NaOH and sodium chloroacetate in the reaction mixture (Table 3). Carboxymethylation efficiency based upon sodium chloroacetate consumption was 86%±1%.

TABLE 3

| | | | | | | | Weight | DS by | Carboxymethylation |
|---|---|---|---|---|---|---|---|---|---|
| CMG | Guar | | NaOH | | SCA | | gain | Titration | efficiency, % |
| | 70.0 | g | 9.92 | g | 24 | g | 21 g | 0.41 | 86 |
| | (0.432 | meq) | (0.248 | meq) | (0.206 | meq) | | | |
| | 140.0 | g | 19.92 | g | 48 | g | 27 g | 0.40574 | 85 |
| | (0.764 | meq) | (0.498 | meq) | (0.412 | meq) | | | |
| | 140.0 | g | 28 | g | 67.76 | g | 50 g | 0.5855 | 87 |
| | (0.864 | meq) | (0.72 | meq) | (0.581 | meq) | | | |

### EXAMPLE 10

### Preparation of Methyl Carboxymethyl Guar (MCMG)

NaCMG or NaCMHPG (20.0-40.0 g) was slurried in 35-50 mL of dimethyl sulfate (DMS). The slurry was stirred for 4-8 hours at 60° C. under nitrogen. The mixture was filtered, washed and soaked with 450 mL of methanol, and then again washed and then soaked with 450 mL of acetone. The filtrate was then dried at 60° C. overnight. The product was insoluble in H₂O. When the methylation was conducted under nitrogen and the temperature was maintained below 60° C., a high conversion to the methyl ester was achieved. The reaction time was shortened by pretreating the NaCMG with DMSO before adding DMS. The derivatives were good substrates onto which polyoxyalkyleneamines could be grafted. The MCMG was used without further purification in subsequent synthesis. [HATR FT-IR (cm⁻¹), solid: MCMG, 3393 (w, O—H), 2913 (w, C—H), 1732 (s, ester carbonyl), 1026 (vs, C—O).]

### EXAMPLE 11

### Controlled Grafting

The technique for grafting oligomers to guar gum was adapted from methods which were developed in our lab to modify carboxymethyl cellulose (CMC) derivatives (ASTM D 1439, Vol. 6.03 (1994)). Conversion of CMC to the corresponding ester afforded a derivative, which was reacted with a diamine to produce a water soluble aminoamide derivative. Conversion of an ester to an amide was achieved in the second step, during which the polyoxyalkyleneamine reacted with the ester. Grafting of MCMG with polyoxyalkyleneamine was monitored using FT-IR. The derivatives had a low degree of substitution.

### EXAMPLE 12

### Reaction of Methyl Carboxymethyl Guar with Polyoxyalkyleneamines

Approximately 20 g of CMG-CH₃ or CMHPG-CH₃ ester was contacted for 15 min with 100 mL of hot DMSO (95° C.). A slight molar excess of polyoxyalkyleneamine was then added and allowed to react for 24-48 hours at 90-95° C. After cooling to room temperature, 100 mL CH₂Cl₂ was added and the slurry was stirred for 15 min before it was filtered. The collected solid was contacted with 100-150 mL CH₂Cl₂ stirred for 15-30 min, filtered, and then washed with acetone twice to remove DMSO and excess polyoxyalkyleneamine. The product was filtered and further dried in an oven at 60° C. overnight. [HATR FT-IR (cm⁻¹), solid: CMG-g-polyoxyalkyleneamine, 3393 (w, O—H), 2913 (w, C—H), 1640 (sh, amide I band), 1594 (s, amide II band) 1066 and 1020 (vs, C—O—C).]

### EXAMPLE 13

The structures of several polyoxyalkyleneamines used in prototype experiments are shown in Table 4. These amino compounds were selected for their relative hydrophobicity and molecular weight. Hydrophobicity was controlled by varying the ratio of propylene oxide (PO) units to ethylene oxide (EO) units in the oligomer.

TABLE 4

| Polyoxyalkyleneamines | | Ratio | Approximate |
|---|---|---|---|
| Name | Structure | PO/EO (y/x) | Mol. Wt. |
| XTJ-505 (M-600) | CH₃—[OCH₂—CH²]ₓ—[OCH₂—CH(CH³)]_{y}—NH₂ | 9/1 | 600 |
| Jeffamine M-715 | CH₃—[OCH₂—CH²]ₓ—[OCH₂—CH(CH³)]_{y}—NH₂ | 2/11 | 715 |
| XTJ-506 (M-1000) | CH₃—[OCH₂—CH²]ₓ—[OCH₂—CH(CH³)]_{y}—NH₂ | 3/19 | 1000 |
| Surfonamine L-300 | CH₃—[OCH₂—CH²]ₓ—[OCH₂—CH(CH³)]_{y}—NH₂ | 8/58 | 3000 |
| Surfonamine MNPA- | C₉H₁₉—C₆H₄—O—[CH₂CH(CH₃)]_{12.5}—OCH₂CH(CH₃)—NH₂ | — | 1004 |
| 1000 (B100) | | | |
| Surfonamine ML-300 | CH₃(CH₂)₁₂OCH₂CH(CH₃)—OCH₂CH(CH₃)—NH₂ | — | 325 |
| (B30) | | | |

### EXAMPLE 14

### Characterization of the Guar Gum Grafted Products

To confirm the presence of grafts on the guar backbone, we analyzed the gel precursors using ¹H NMR, and calculated the percent grafting from integration of the spectra. All ¹H NMR spectra showed characteristic peaks for the graft and the guar backbone. The ¹H NMR of base CMG contained no peaks in the region 0.5-1.5 ppm. The ¹H NMR of CMG-M1000 had two doublets representing the —CH₃ located at each end group (0.927 and 0.949 ppm) and the —CH₃ located at the propylene oxide within the chain (1.062 and 1.082 ppm). In addition, the spectrum showed a very strong peak at 3.621 ppm (—CH₂—O—), attributed to the protons of the oxyethylene and oxypropylene units within the backbone of the chains. The ¹H NMR spectrum of CMG-M1000 showed the two doublet peaks at 0.927 and 0.949 ppm and the strong peak at 3.614 ppm with a small upfield shift. The characteristic peaks for the guar backbone appeared as a broad multiplet from 3.5-4.0 ppm. The existence of the characteristic peaks of the M-1000 in the grafted product indicated a successful grafting process.

Grafts of CMG with polyoxyalkyleneamines M-600, M-715, and L300 showed similar ¹H NMR spectra to the CMG-M1000. The spectrum of grafts for CMG with MNPA-1000 and B30 showed more unique features associated with the alkylaryloxy and lauryloxy end groups respectively. The ¹H NMR spectrum of CMHPG-M600 illustrated that similar grafted structures were prepared from carboxymethyl hydroxypropyl derivatives, but the unique resonances of M600 both in the 0.5-1.5 ppm and the 3.4-3.8 ppm regions of the spectra overlapped with the resonances associated with the hydroxypropyl substituents. The unique resonances can be detected, but accurate integration of the signals could not be achieved. Thus, values for the percentage grafting to carboxymethyl hydroxypropyl derivatives (Table 5) are estimates.

### EXAMPLE 15

### Determination of Graft Percentage by NMR

The percent grafting in the product CMG-M1000 was determined by analyzing the two major regions both in the ¹HNMR spectra of the CMG control gel, the polyoxyalkyleneamine (M1000), and in graft (CMG-M1000): one peak at 0.5-1.5 ppm, and one at 2.8-4.5 ppm. An internal standard of 1% sodium-3-trimethylsilylpropionate-2,2,3,3-d₄ was used to calibrate the integration. Then m, the percent grafting, was calculated using the relative areas of the graft and backbone resonances.

### EXAMPLE 16

Tables 5 and 6 depict results from the syntheses of several grafted CMHPGs and CMGs. Grafted products were not recovered quantitatively. While not wishing to be bound by this theory, it appears that the losses were due to wide distributions of molecular weights of the substrates, and to non-homogeneous distributions of carboxymethyl substituents on the guar molecules. Further, lower molecular weight molecules with higher DS were difficult to isolate from DMSO and the washing solvents. The isolated yields of the grafted materials ranged from 17-71%. The lowest yields were obtained with the highest molecular weight amine, surfonamine L-300, suggesting that reaction may have been inhibited by poor accessibility of the amine functional group.

TABLE 5
Percent yield, degree of substitution (DS), and
percent grafting to CMHPG products.

| | | Weight | | | |
|---|---|---|---|---|---|
| | Wt. of | of isolated | | % | % Graft |
| Gel Name | Polymer | product | DS | Yield | (m) |
| CMHPG-M600 | 20 | 18.98 | 0.25 | 51.76 | 1.80 |
| CMHPG-M715 | 20 | 21.37 | 0.25 | 53.61 | 5.02 |
| CMHPG-M1000 | 20 | 18.81 | 0.25 | 39.37 | 11.95 |
| CMHPG-MNPA1000 | 20 | 20.20 | 0.25 | 42.18 | 8.38 |
| CMHPG-L300 | 20 | 18.95 | 0.25 | 18.34 | 7.87 |
| CMHPG-B30 | 20 | 18.62 | 0.25 | 64.15 | 28.29 |
| CMHPG-M600 | 20 | 19.64 | 0.18 | 60.73 | 0.43 |
| CMHPG-M715 | 20 | 16.39 | 0.18 | 47.22 | 1.66 |
| CMHPG-M1000 | 20 | 19.96 | 0.18 | 49.19 | <0.01 |
| CMHPG-MNPA1000 | 18 | 17.90 | 0.18 | 48.92 | 7.35 |
| CMHPG-L300 | 20 | 18.38 | 0.18 | 22.49 | 0.03 |
| CMHPG-B30 | 20 | 18.84 | 0.18 | 70.60 | 6.67 |

TABLE 6
Percent yield, degree of substitution (DS) and
percent grafting to CMG products.

| | Wt. of | Weight of | | % | % Graft |
|---|---|---|---|---|---|
| Gel Name | Polymer | isolated product | DS | Yield | (m) |
| CMG-M600 | 20 | 19.19 | 0.28 | 49.92 | 2.16 |
| CMG-M1000 | 20 | 19.76 | 0.28 | 38.94 | 2.65 |
| CMG-MNPA1000 | 22.1 | 22.2 | 0.28 | 39.50 | 9.07 |
| CMG-L300 | 16 | 15.53 | 0.28 | 17.30 | 2.83 |
| CMG-B30 | 16.25 | 15.93 | 0.28 | 65.37 | 7.71 |
| CMG-M715 | 18 | 19.15 | 0.41 | 42.04 | 6.88 |
| CMG-M600 | 18 | 16.6 | 0.41 | 40.37 | 4.70 |
| CMG-M1000 | 18 | 16.4 | 0.41 | 29.01 | 4.37 |
| CMG-MNPA1000 | 18 | 18.23 | 0.41 | 32.16 | 24.43 |

### EXAMPLE 17

### Viscosity Determination

The viscosities of the grafted CMG and CMHPG gel precursors were compared with the viscosity of corresponding control gel precursors, CMG and CMHPG. A drop in viscosity of the grafted products relative to that of the control polymers indicated the surfactant effect of the introduced graft.

Solutions of the grafted products were prepared at high pH (>9.5) because of higher solubility. Table 7 shows the Brookfield viscosity at 20 rpm of the control materials compared to their corresponding grafted derivatives. The table illustrates that grafted materials had lower viscosities than the parent materials. In addition, the hydrophobic (M600, MNPA1000, and B30) grafts showed slightly higher viscosities than the hydrophilic grafts. The B30 graft, which was the most hydrophobic graft, exhibited the highest viscosity.

TABLE 7
Brookfield viscosity of the control polymer
compared to grafted derivatives

| | | | Viscosity |
|---|---|---|---|
| Gel Name | Concentration | pH | cP (mPa · s) at 20 rpm |
| CMHPG | 1% | 9.00 | 4640 |
| CMHPG-M600 | 1% | 7.19 | 120 |
| CMHPG-M715 | 1% | 6.6 | 40 |
| CMHPG-M1000 | 1% | 6.7 | 90 |
| CMHPG-MNPA1000 | 1% | 5.6 | 1310 |
| CMHPG-L300 | 0.48% | 10.25 | 23 |
| CMHPG-B30 | 0.48% | 10.23 | 43 |
| CMHPG | 1% | 9.24 | 3200 |
| CMHPG-M600 | 1% | 8.3 | 290 |
| CMHPG-M715 | 1% | 7.8 | 75 |
| CMHPG-M1000 | 1% | 8.42 | 260 |
| CMHPG-MNPA1000 | 1% | 6 | 95 |
| CMHPG-L300 | 1% | 5.7 | 195 |
| CMHPG-B30 | 0.48% | 10.39 | 90 |
| CMG | 0.48% | 10 | 310 |
| CMG-M600 | 0.48% | 10 | 55 |
| CMG-M1000 | 0.48% | 10 | 40 |
| CMG-MNPA1000 | 0.48% | 10 | 65 |
| CMG-L300 | 0.48% | 10 | 45 |
| CMG-B30 | 0.48% | 10.26 | 75 |

### EXAMPLE 18

### Carboxymethylated Guar Derivatives (CMG)

The viscosities of grafted derivatives of CMG were lower at low shear rates than that of the CMG control gel, but all gel precursors showed the same shear thinning behavior. The control polymer solutions continued to shear thin throughout the range of shear rates measured. At shear rates above 11.3 s⁻¹, solutions of all derivatives retained higher viscosities than those of the control polymer. The thickening at the intermediate shear rates appeared to be related to the history of solution preparation. However, for the second runs, this thickening seemed to disappear. While not wishing to be bound by this theory, it appears that the different behavior of the grafted gel precursors was related to large side chains added during the grafting process. It appears that the side chains required at least one run, or high shear, to align, after which they no longer contributed to the observed thickening.

### EXAMPLE 19

### Gel Formation

Polymer solutions were prepared by dispersing the polymers in deionized water in concentrations from about 4.8 g/L (40 lb/1000 gal) to about 2.4 g/L (20 lb/1000 gal). The gels were allowed to hydrate for at least 30 min. A small amount of sodium thiosulfate at a concentration of 1.2 g/L (10 lb/1000 gal) was added as a gel stabilizer. We also used deionized water containing 4.61×10⁻⁴ g/L sodium azide to stop microorganism growth in some cases.

Prior to testing, the pH was adjusted to >10 with sodium carbonate at a concentration of 0.6 g/L (5 lb/1000 gal). The dispersion was stirred until all the sodium carbonate dissolved. The linear gel precursor was transferred to a Waring blender, and under conditions of excessive shear, 0.3 mL-0.5 mL of zirconium lactate (cross-linking agent; Benchmark, 8.3% ZrO) was added. Blending was continued until the vortex disappeared. The resulting gel was transferred to a heatable sample cup, and the viscosity was measured at different shear rates at room temperature (25° C.).

### EXAMPLE 20

Using the methods and reagents described above, the following gels were made: CMG (Control), CMG-M600, CMG-M1000, CMG-L300, CMG-MNPA1000, CMG-B30, CMHPG-M600, CMHPG-M715, CMHPG-M1000, CMHPG-MNPA1000, CMHPG-L300, and CMHPG-B30.

### EXAMPLE 21

All derivatives were successfully crosslinked using a zirconium lactate crosslinking agent at a concentration of 40 lb/1000 gal (4.8 g/L), which is above the critical concentration for gelation, C_{cc}, for both CMG and CMHPG. At a concentration of 20 lb/1000 gal (2.4 g/L), which is very near C_{cc} for CMHPG and slightly above C_{cc} for CMG, the crosslinking was successful for all the CMG derivatives, but successful for only the B30 and M600 derivatives of CMHPG.

### EXAMPLE 22

Table 8 shows the average viscosities of the cross-linked CMG control gel, with a concentration of 20 lb. per 1000 gal water (2.4 g/L) (“20 gel”), compared to its grafted derivatives. The L300, M1000, and MNPA1000 derivatives had initial viscosities in the range of 1500-1900 cP at room temperature (“RT”). The viscosities of these gels decreased at 65° C. to an average of 220-280 cP. The second gel group, which included the control, M600, and B30 gels, showed lower initial viscosities at RT in the range of 550-750 cP. These gels tended to retain a larger fraction of their viscosities upon heating to 65° C. (380-460 cP). The first group lacked acceptable thermal stability at this concentration. At the 20 gel concentration, the mixtures only marginally supported crosslinking. Some polymers crosslinked and some did not.

TABLE 8
Average viscosities of 20-gel CMG control gel and its derivatives
(shear rate of 37.7/s, 2.4 g/L, pH > 10, 0.4 mL of Zr cross-linking agent)

| | Initial Viscosity | Avg. Viscosity | Std. Dev. |
|---|---|---|---|
| Gel name | at R.T., cP | at 65° C., cP | (“SD”) |
| CMG Control | 550 | 460 | 72 |
| CMG-M600 | 750 | 380 | 6 |
| CMG-M1000 | 1940 | 250 | 9 |
| CMG-L300 | 1520 | 280 | 12 |
| CMG-MNPA1000 | 1800 | 220 | 11 |
| CMG-B30 | 750 | 440 | 25 |

### EXAMPLE 23

Table 9 shows average viscosities for the cross-linked CMG control gel at a shear rate of 37.7 s⁻¹ at a concentration of 40 lb. per 1000 gal water (4.8 g/L) (40 gel) compared to gels prepared from its corresponding grafted derivatives. At the 40 gel concentration, crosslinking was more consistent. The gels were divided into two groups according to their average viscosities at 65° C. The control, M1000, M600, and MNPA1000 derivatives had initial viscosities at room temperature (RT) of 3410, 1580, 1020, and 800 cP, respectively. These gels showed a decrease in viscosities at 65° C. to 590-920 cP.

TABLE 9
Average viscosities of 40-gel CMG control gel and its derivatives
(shear rate of 37.7/s, 4.8 g/L, pH > 10, 0.5 mL of Zr cross-linking agent).

| | Initial Viscosity | Avg. Viscosity | |
|---|---|---|---|
| Gel name | at R.T., cP | at 65° C., cP | SD |
| CMG Control | 3410 | 760 | 13 |
| CMG-M600 | 1020 | 920 | 63 |
| CMG-M1000 | 1580 | 590 | 36 |
| CMG-L300 | 2220 | 5410 | 706 |
| CMG-MNPA1000 | 800 | 670 | 22 |
| CMG-B30 | 1940 | 6660 | 1239 |

### EXAMPLE 24

The second group shown in Table 9 included L300 and B30 gels. This group showed lower viscosities at room temperature than that of the control gel. However, the viscosities of these gels increased at 65° C., and were nearly an order of magnitude higher than those of the control gel. At the 40-gel concentration, all gels appeared relatively stable to long term shear and exposure to the elevated temperature. L300 and B30 grafted gels showed superior characteristics for fracturing fluid applications, based upon their high viscosities during the aging period. These samples differed substantially from each other. The L300 gel, which had a high molecular weight (3000 Daltons) hydrophilic graft, was obtained in low percentage (2.83%) yield. In contrast, the B30 gel, which had a low molecular weight and was very hydrophobic with a long alkyl chain tail, incorporated at a higher percentage yield (7.71%).

### EXAMPLE 25

Table 10 shows the average viscosities of the crosslinked CMG control gel (20 gel) compared to its grafted derivatives at a continuous shear rate of 37.7 s⁻¹ at two different temperatures. The table lists the average viscosities recorded over the last 60-80 minutes of measurement. For each measurement, 30-60 minutes were required to reach temperature equilibrium in the gel. At low concentrations, we observed two groups of gels based on average viscosities at 65° C. and 90° C.

The L300, M1000, and MNPA1000 derivatives had initial viscosities at room temperature (RT) in the range of 1500-1900 cP. These gels showed a decrease in average viscosity at 65° C. to 200-250 cP. The average viscosities of these gels increased at 90° C. to 250-430 cP. While not wishing to be bound by this theory, it appears that the increase in viscosity can be attributed to the loss of solvent (water) from the open cup system. The other group, which included the control, M600, and B30 gels, showed lower initial viscosities at RT, 550-750 cP. These gels possessed higher average viscosities than the first group at 65° C. (370-450 cP). At 90° C., the average viscosities of these gels increased to 560-675 cP. The increased viscosity of the derivative gels with temperature may also be attributed to loss of water from the systems.

TABLE 10
Average viscosity of crosslinked CMG control gel and its derivatives
(shear rate of 37.7/s, 2.4 g/L, pH > 10,
0.4 mL of Zr crosslinking agent).

| | | | | Avg. | |
|---|---|---|---|---|---|
| | Initial | Avg. | | Viscosity | |
| | Viscosity | Viscosity at | | at | |
| Gel name | at R.T., cP | 65° C., cP | SD | 90° C., cP | SD |
| CMG Control | 548 | 465.1 | 72.6 | 673.6 | 47.4 |
| CMG-M600 | 748 | 383.3 | 6.7 | 568.3 | 52.5 |
| CMG-M1000 | 1939 | 248.1 | 9.7 | 314.2 | 36.5 |
| CMG-L300 | 1524 | 279.3 | 12.4 | 432.9 | 53 |
| CMG-MNPA1000 | 1805 | 219.9 | 11.8 | 257.4 | 18.5 |
| CMG-B30 | 748 | 439.8 | 25.4 | 575 | 10.1 |

### EXAMPLE 26

Table 11 shows the average viscosities of the crosslinked CMG control (40 gel concentration) compared to grafted gels at a shear rate of 37.7 s⁻¹. We observed two groups of gels based on average viscosities at 65° C. and 90° C. The control, M1000, M600, and MNPA1000 derivatives had initial viscosities at room temperature (RT) of 3409, 1577, 1016, and 802 cP. The gels showed a decrease in average viscosity at 65° C. (590 to 930 cP). The average viscosities of these gels at 90° C. (640-900 cP) showed little change.

The other group, which included L300 and B30 gels, showed initial viscosities at RT of 2219 and 1939 cP. These gels showed higher average viscosities at 65° C. of 5337 and 6664 cP respectively. At 90° C., the average viscosities of these gels decreased. In general, the first group had lower, but more stable, average viscosities. The second group had higher, but more unstable, viscosities at lower temperature (65° C.), and more stable viscosities at 90° C.

TABLE 11
Average viscosity of crosslinked CMG control gel and its derivatives
(shear rate of 37.7/s, 4.8 g/L, pH > 10,
0.5 mL of Zr crosslinking agent).

| | Initial | Avg. | | Avg. | |
|---|---|---|---|---|---|
| | Viscosity | Viscosity | | Viscosity | |
| | at | at | | at | |
| Gel name | R.T., cP | 65° C., cP | SD | 90° C., cP | SD |
| CMG Control | 3409 | 756.3 | 13.6 | 698.7 | 59.7 |
| CMG-M600 | 1016 | 922.7 | 63.1 | 901 | 88.7 |
| CMG-M1000 | 1577 | 594.3 | 36.7 | 678.7 | 46.7 |
| CMG-L300 | 2219 | 5411.4 | 706.7 | 4901 | 121.9 |
| CMG-MNPA1000 | 802 | 671.6 | 22 | 646.9 | 51.5 |
| CMG-B30 | 1939 | 6664 | 1239 | 5783.6 | 383 |

### EXAMPLE 27

### Carboxymethylhydroxypropyl Guar Derivatives (CMHPG)

The effect of grafts on gel viscosities was modulated by the presence of hydroxylpropyl substituents. At room temperature, the viscosity of the crosslinked CMHPG control (20 gel concentration) showed the same shear thinning behavior as its grafted derivatives. The control, B30, and M600 gels showed higher viscosities than other derivatives at all shear rates. The M1000 derivative showed an intermediate viscosity at room temperature. The viscosities of CMHPG crosslinked gels (40 gel concentration) showed the same shear thinning behavior for all shear rates. The M600 and B30 gels showed higher viscosities than the other gels at most shear rates.

### EXAMPLE 28

Table 12 shows the average viscosities of the crosslinked gels (20 gel concentration) for the CMHPG control gel and its derivatives at a continuous shear rate of 37.7 s⁻¹. Most samples at the 20 gel concentration of the CMHPG grafted samples did not produce strong gels. Introduction of the grafts increased the critical cross-linking concentration. Only the B30 and M600 samples performed well under these conditions. At this concentration three gels (M715, MNPA1000, and L300) failed to crosslink to form a gel at all. However, at RT M1000, M600, B30, and the control gels exhibited initial viscosities between 260 and 415 cP. Raising the temperature to 65° C. caused all viscosities to decrease. At 90° C. all gels showed increased viscosities.

TABLE 12
Average viscosity of crosslinked CMHPG control gel and its derivatives
(shear rate of 37.7/s, 2.4 g/L, pH > 10, 0.4 mL of Zr crosslinking agent).

| | Initial | Avg. | | Avg. | |
|---|---|---|---|---|---|
| | Viscosity | Viscosity at | | Viscosity at | |
| Gel name | at R.T. | 65° C. | SD | 90° C. | SD |
| CMHPG Control | 414 | 291.2 | 20.9 | 367.8 | 66.5 |
| CMHPG-M600 | 401 | 294.2 | 29.9 | 519.9 | 52.7 |
| CMHPG-M715 | ND | ND | ND | ND | ND |
| CMHPG-M1000 | 267 | 147.1 | ND | 150.1 | 5.9 |
| CMHPG-L300 | ND | ND | ND | ND | ND |
| CMHPG- | ND | ND | ND | ND | ND |
| MNPA1000 | | | | | |
| CMHPG-B30 | 361 | 312 | 18.9 | 361.1 | 31.2 |

### EXAMPLE 29

Table 13 shows the average viscosities of the crosslinked CMHPG control and its grafted gels at shear rate of 37.7 s⁻¹, at 40 gel concentrations. The control, M1000, and L300 derivatives had initial viscosities at room temperature of 655, 561, and 468 cP, respectively. These gels showed a decrease in viscosity at 65° C. At 90° C. the average viscosities of these gels showed a slight increase for M1000, a decrease for the control, and little change for L300.

The other group included the M600, M715, MNPA1000, and B30 derivatives. These derivatives showed initial viscosities at RT of 1270, 615, 414 and 802 cP, respectively. The M600 and B30 gels had lower average viscosities at 65° C., but at 90° C. their average viscosities increased. The average viscosities of the M715 and MNPA1000 increased at 65° C., and at 90° C. the average viscosity for MNPA1000 increased slightly, while the viscosity for M715 decreased slightly.

TABLE 13
Average viscosity of crosslinked CMHPG control gel and its derivatives
(shear rate of 37.7/s, 4.8 g/L, pH > 10, 0.5 mL of Zr crosslinking agent).

| | Initial | Avg. | | Avg. | |
|---|---|---|---|---|---|
| | Viscosity | Viscosity at | | Viscosity at | |
| Gel name | at R.T. | 65° C. | SD | 90° C. | SD |
| CMHPG Control | 655 | 419 | 6.7 | 202.1 | 36.9 |
| CMHPG-M600 | 1270 | 537.9 | 33.3 | 575 | 13.4 |
| CMHPG-M715 | 615 | 793.4 | 39.6 | 772.3 | 129.8 |
| CMHPG-M1000 | 561 | 194.6 | 22.3 | 372.9 | 51.1 |
| CMHPG-L300 | 468 | 271.9 | 13.4 | 280.8 | 72.9 |
| CMHPG- | 414 | 610.7 | 90.9 | 674.6 | 102.3 |
| MNPA1000 | | | | | |
| CMHPG-B30 | 802 | 475.5 | 34.2 | 824.1 | 275.3 |

### EXAMPLE 30

### Viscosity Measurements at Elevated Temperature and Pressure

The viscosity of the hydrophobic MNPA1000 and B30 derivatives was tested at elevated temperature and pressure to simulate conditions in geological formations.

The three control gels showed low viscosity (220-250 cp). While not wishing to be bound by this theory, we believe such low viscosities were due to the inhomogeneous gels that resulted from the rapid cross-linking (2-5 s). The rapid cross-linking may not have allowed the cross-linking agent to be distributed evenly in the material to form a homogeneous network. Viscosity of CMG when subjected to 90° C. and 120° C. for at least two hours was low but stable. When subjected to the same heating protocol, the B30 and MNPA1000 derivatives each exhibited higher initial viscosities than the control. The gel produced from the B-30 derivative exhibited an initial viscosity of 1900 cP. This viscosity dropped upon aging at 90° C. to 1600 cP. Heating this gel to 120° C. led to a steady degradation and corresponding reduction in viscosity. The final viscosity was 600 cP, which still exceeded the viscosity of the control. Similar aging trends were observed with the gel derived from the MNPA derivative. While not wishing to be bound by this theory, it appears that the superior performance of these derivatives may be due to a well organized and homogeneous network produced from the slow gelling time (20-30 s).

### EXAMPLE 31

### Gel Hydrolysis

One percent aqueous solutions were prepared from the control and the modified gels. Some of the gels were cross-linked using a zirconium agent. After the pH of the fluids was adjusted to between 5-9, 0.3 mL of the enzyme breaker (Pyrolase™ 200) was added to approximately 100 mL of solution/gel at a temperature of between 55-60° C. After 2 hours, the mixture was cooled to room temperature. The effectiveness of breaking and the hydrophobicity of the fragments were shown by extracting these fragments in 15 to 25 mL aliquots of toluene. The gels were stirred using a vortex stirrer and then allowed to separate by phase. For some of the gels, the quantity of the broken graft fragments was measured by evaporating the solvents from the separate layers and then analyzing the residue with FT-IR and matrix assisted laser desorption/ionization mass spectroscopy (MALDI-MS) to identify the components. Evidence for poly(oxylalkylene)amides coupled to oligosaccharide fragments was observed.

Miscellaneous

The complete disclosures of all references cited in this specification are hereby incorporated by reference. Also incorporated by reference is the complete disclosure of W. Daly et al., “Poly(oxyalkylene) grafts to guar gum with applications in hydraulic fracturing fluids,” abstract available online approx. July 2005 at www.bme.hu/pat2005/, presented on Sep. 13, 2005 at the 8^{th} International Symposium, Polymers for Advanced Technologies, Budapest. In the event of an otherwise irreconcilable conflict, however, the present specification shall control.

What is claimed:

1. A substituted polysaccharide selected from the group consisting of guar, cellulose, starch, chitin, chitosan, polygalactomannan, polyglucomannan, galactomannan gum, xanthan, and derivatives of these compounds; wherein said polysaccharide comprises a plurality of repeating monosaccharide units or repeating oligosaccharide units; and wherein between about 2% and about 50% of said repeating units are substituted with an alkoxyetheramide selected from the group consisting of alkylaryloxypoly(oxyalkylene)amides and alkoxypoly(oxyalkylene)amides.

2. A compound as recited in claim 1 having the structure: 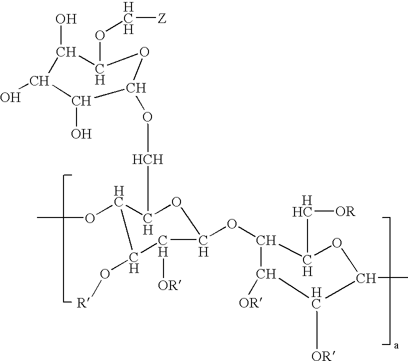
wherein:
Z is selected from the group consisting of H, C₉H₁₉—C₆H₄—O—[CH₂CH(CH₃)]_{12.5}—OCH₂CH(CH₃)—NHC═O, CH₃(CH₂)₁₂OCH₂CH(CH₃)—OCH₂CH(CH₃)—NHC═O, and 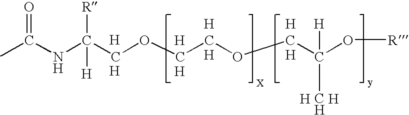
wherein the percentage of substitution of Z onto the polysaccharide backbone when Z is not H is between about 2% and about 50%;
R is selected from the group consisting of —H, —CH₂CH(OH)CH₃, and —CH₂COO⁻M⁺;
R′ is selected from the group consisting of —H and —CH₂CH(OH)CH₃;
The R′ groups may be the same or different;
M⁺ is selected from the group consisting of H⁺, (NR₄″)⁺, Li⁺, Na⁺, K⁺, Rb⁺, and Cs⁺;
a is an integer between 100 and 10,000;
x is an integer between 1 and 60;
y is an integer between 1 and 15;
R″ is —H or —CₘH₂ₘ₊₁;
m is an integer from 1 to 20;
R′″ is —H, —CₙH₂ₙ₊₁, or Ar—CₙH₂ₙ₊₁;
Ar is C₆H₄, C₁₀H₈ or C₁₄H₁₂; and
n is an integer from 1 to 20.

3. A process for forming a viscous gel, and then, at a time selected by a user, fragmenting the gel into fragments with surfactant properties; said process comprising the steps of:
(a) converting a low-viscosity aqueous solution of a compound as recited in claim 1 having a viscosity less than about 100 cP, into a high-viscosity aqueous gel, having a viscosity greater than about 800 cP; wherein said converting step comprises crosslinking said compounds in aqueous solution; and
(b) at a time selected by a user, fragmenting the gel into substituted oligosaccharide fragments having surfactant properties, forming an aqueous emulsion of substituted oligosaccharides having a viscosity below about 10 cP.

4. A process as recited in claim 3 wherein the low-viscosity aqueous solution has a viscosity between about 30 cP and about 100 cP; and wherein the high-viscosity aqueous gel has a viscosity greater than about 2000 cP.

5. A process as recited in claim 3 wherein the percentage of substitution is between about 15% and about 25%.

6. A process as recited in claim 3 wherein said crosslinking comprises reacting the polymers with a crosslinking agent selected from the group consisting of boric acid, borate salts, zirconates, ZrOCl₂, zirconium lactate, zirconium glycolate, zirconium lactate triethanolamine, zirconium acetylacetonate, Zr chelates, titania, titanates, titanium citrate, titanium malate, titanium tartrate, Ti chelates, and aluminates.

7. A process as recited in claim 3 wherein said fragmenting step comprises reacting the high-viscosity aqueous gel with one or more breaking agents selected from the group consisting of peroxides, persulfates, perborates, oxyacids, oxyanions of halogens, Cu⁺²-chelated EDTA, aminocarboxylates, diamines, FeCl₂ and FeCl₃.

8. A process as recited in claim 3 wherein said fragmenting step comprises reacting the high-viscosity aqueous gel with one or more hydrolytic enzymes.

9. A process as recited in claim 3 wherein the one or more hydrolytic enzymes are selected from the group consisting of cellulases, amylases, guarases, and chitinases.

10. A process as recited in claim 3 wherein said fragmenting step comprises incorporating into the high-viscosity aqueous gel one or more delayed breaking agents, and allowing the delayed breaking agent to fragment the gel with the passage of time.

11. A process as recited in claim 10 wherein the one or more delayed breaking agents are selected from the group consisting of metaperiodic acid, metaperiodic acid salts, potassium metaperiodate, sodium metaperiodate, ammonium metaperiodate, calcium metaperiodate, and lithium metaperiodate.

12. A process as recited in claim 11 additionally comprising the steps of:
(a) hydraulically fracturing a rock formation with the high-viscosity aqueous gel, wherein the gel additionally contains suspended proppant particles; and
(b) removing the surfactant molecules in an aqueous emulsion from the fractured rock formation, while leaving proppant particles within the fractured rock formation to help hold open cracks in the formation caused by the hydraulic fracturing.

13. A process for delivering a pharmaceutical composition to a human retina in vivo in a time-released manner; said process comprising the steps of:
(a) converting a low-viscosity aqueous system into a high-viscosity aqueous gel having a viscosity greater than about 800 cP; wherein:
(i) the low-viscosity aqueous system comprises a pharmaceutical composition and a compound as recited in claim 1;
(ii) the low-viscosity aqueous system has a viscosity less than about 100 cP;
(iii) said converting step comprises crosslinking polymers in the aqueous system; wherein, prior to said crosslinking, the polymers comprise one or more uncharged, water-soluble polysaccharides that are substituted with one or more hydrophobic substituents, with a percentage of substitution between about 2% and about 50%, and wherein the gel contains the pharmaceutical composition; and
(iv) said converting step comprises injecting the low-viscosity aqueous system and a crosslinking agent onto the retina or in the immediate vicinity of the retina, so that the crosslinking agent and the low-viscosity aqueous system react on the retina or in the immediate vicinity of the retina to form in situ a high-viscosity aqueous gel containing the pharmaceutical composition on the retina or in the immediate vicinity of the retina; and
(b) allowing the gel to remain on or in the immediate vicinity of the retina, and to release the pharmaceutical composition over a period of time.

14. A process as recited in claim 13 additionally comprising the step of fragmenting the gel, at a time selected by a user, into substituted oligosaccharide fragments having surfactant properties, forming an aqueous emulsion of substituted oligosaccharides having a viscosity below about 10 cP; and removing the surfactant molecules and the pharmaceutical composition from the retina or from the immediate vicinity of the retina.

15. A process for forming a viscous gel, and then, at a time selected by a user, fragmenting the gel into fragments with surfactant properties; said process comprising the steps of:
(a) converting a low-viscosity aqueous solution, having a viscosity less than about 100 cP, into a high-viscosity aqueous gel, having a viscosity greater than about 800 cP; wherein said converting step comprises crosslinking polymers in aqueous solution; wherein, prior to said crosslinking, the polymers comprise one or more uncharged, water-soluble polysaccharides that are substituted with one or more hydrophobic substituents, at a percentage of substitution between about 2% and about 50%, and wherein the hydrophobic substituents have a molecular weight between about 250 Dalton and about 3000 Dalton; and
(b) at a time selected by a user, fragmenting the gel into substituted oligosaccharide fragments having surfactant properties, thus forming an aqueous emulsion of substituted oligosaccharides having a viscosity below about 10 cP; wherein the hydrophobic substituents form the hydrophobic segments of the surfactant molecules, and wherein the oligosaccharide portions form the hydrophilic segments of the surfactant molecules.

16. A process as recited in claim 15 wherein the low-viscosity aqueous solution has a viscosity between about 30 cP and about 100 cP; and wherein the high-viscosity aqueous gel has a viscosity greater than about 2000 cP.

17. A process as recited in claim 15 wherein the percentage of substitution is between about 15% and about 25%, and wherein the hydrophobic substituents have a molecular weight between about 300 Dalton and about 1000 Dalton.

18. A process as recited in claim 15 wherein the polymers prior to said crosslinking are one or more substituted polysaccharides or polyvinyl alcohols, wherein the polysaccharides are selected from the group consisting of a modified guar powder, carboxymethyl guar, cellulose, starch, chitin, chitosan, polygalactomannan, polyglucomannan, galactomannan gum, xanthan, guar gum, locust bean gum, honey locust gum, flame tree gum, Cassia occidentialis gum, karaya gum, carragenan, hydroxypropyl guar, carboxymethyl hydroxypropyl guar, or carboxymethyl guar, and derivatives of any of the foregoing.

19. A process as recited in claim 15 wherein the one or more hydrophobic substituents are selected from the group consisting of alkylaryloxypoly(oxyalkylene)amides and alkyloxypoly(oxyalkylene)amides.

20. A process as recited in claim 15 wherein said crosslinking comprises reacting the polymers with a crosslinking agent selected from the group consisting of boric acid, borate salts, zirconates, ZrOCl₂, zirconium lactate, zirconium glycolate, zirconium lactate triethanolamine, zirconium acetylacetonate, Zr chelates, titania, titanates, titanium citrate, titanium malate, titanium tartrate, Ti chelates, and aluminates.

21. A process as recited in claim 15 wherein said fragmenting step comprises reacting the high-viscosity aqueous gel with one or more breaking agents selected from the group consisting of peroxides, persulfates, perborates, oxyacids, oxyanions of halogens, Cu⁺²-chelated EDTA, aminocarboxylates, diamines, FeCl₂ and FeCl₃.

22. A process as recited in claim 15 wherein said fragmenting step comprises reacting the high-Viscosity aqueous gel with one or more polysaccharidases.

23. A process as recited in claim 22 wherein the one or more polysaccharidases are selected from the group consisting of cellulases, amylases, guarases, and chitinases.

24. A process as recited in claim 15 wherein said fragmenting step comprises incorporating into the high-viscosity aqueous gel one or more delayed breaking agents, and allowing the delayed breaking agent to fragment the gel with the passage of time.

25. A process as recited in claim 24 wherein the one or more delayed breaking agents are selected from the group consisting of metaperiodic acid, metaperiodic acid salts, potassium metaperiodate, sodium metaperiodate, ammonium metaperiodate, calcium metaperiodate, and lithium metaperiodate.

26. A process as recited in claim 25 additionally comprising the steps of:
(a) hydraulically fracturing a rock formation with the high-viscosity aqueous gel, wherein the gel additionally contains suspended proppant particles; and
(b) removing the surfactant molecules in an aqueous emulsion from the fractured rock formation, while leaving proppant particles within the fractured rock formation to help hold open cracks in the formation caused by the hydraulic fracturing.

27. A process for delivering a pharmaceutical composition to a human retina in vivo in a time-released manner; said process comprising the steps of:
(a) converting a low-viscosity aqueous system into a high-viscosity aqueous gel having a viscosity greater than about 800 cP; wherein:
(i) the low-viscosity aqueous system comprises a pharmaceutical composition;
(ii) the low-viscosity aqueous system has a viscosity less than about 100 cP;
(iii) said converting step comprises crosslinking polymers in the aqueous system; wherein, prior to said crosslinking, the polymers comprise one or more uncharged, water-soluble polysaccharides that are substituted with one or more hydrophobic substituents, at a percentage of substitution between about 2% and about 50%, and wherein the hydrophobic substituents have a molecular weight between about 250 Dalton and about 3000 Dalton; wherein the gel contains the pharmaceutical composition; and
(iv) said converting step comprises injecting the low-viscosity aqueous system and a crosslinking agent onto the retina or in the immediate vicinity of the retina, so that the crosslinking agent and the low-viscosity aqueous system react on the retina or in the immediate vicinity of the retina to form in situ a high-viscosity aqueous gel containing the pharmaceutical composition on the retina or in the immediate vicinity of the retina; and
(b) allowing the gel to remain on or in the immediate vicinity of the retina, and to release the pharmaceutical composition over a period of time.

28. A process as recited in claim 27 additionally comprising the step of fragmenting the gel, at a time selected by a user, into substituted oligosaccharide fragments having surfactant properties, thus forming an aqueous emulsion of substituted oligosaccharides having a viscosity below about 10 cP; wherein the hydrophobic substituents form the hydrophobic segments of the surfactant molecules, and wherein the oligosaccharide portions form the hydrophilic segments of the surfactant molecules; and removing the surfactant molecules and the pharmaceutical composition from the retina or from the immediate vicinity of the retina.
